(19) United States
(12) Patent Application Publication
(10) Pub. No.: US 2026/0124124 A1
(43) Pub. Date: May 7, 2026

(54) Hair Treatment Compositions

(71) Applicant: TMPL 8 Corp, Miami, FL (US)

(72) Inventor: Kelly Shivangi Van Gogh, Miami, FL (US)

(21) Appl. No.: 19/340,757

(22) Filed: Sep. 25, 2025

Related U.S. Application Data
(60) Provisional application No. 63/699,034, filed on Sep. 25, 2024.

(51) Int. Cl. A61K 8/14; A61K 8/44; A61K 8/49; A61K 8/67; A61K 8/9789; A61K 8/9794; A61Q 5/02

(52) U.S. Cl. CPC A61K 8/14; A61K 8/447; A61K 8/4953; A61K 8/675; A61K 8/9789; A61K 8/9794; A61Q 5/02; A61K 2800/52; A61K 2800/805

(57) ABSTRACT

Hair treatment compositions are disclosed. In some embodiments, the composition can include a first fraction of lipid vesicles encapsulating a first material and a second fraction of lipid vesicles encapsulating a second, different material. In some embodiments, the first material is a stimulant material, such as caffeine or green tea catechins, and the second material is a botanical bioactive material. The lipid vesicles can be plant-derived extracellular vesicles. In some embodiments, the composition comprises a base material with a non-encapsulated stimulant material and lipid vesicles that encapsulate a botanical bioactive material. The composition can also include three distinct carrier systems: lipid vesicles, liposomes, and ceramide complexes, each encapsulating a different bioactive material. These compositions can be formulated into products such as serums, masks, shampoos, or conditioners, which can be part of a multi-product hair care system.

### TECHNICAL FIELD

This relates to hair treatment compositions and, more particularly, to hair treatment compositions including lipid vesicles for delivering bioactive materials.

### BACKGROUND

Hair loss and thinning affect a significant portion of the population, with causes ranging from genetic factors to stress-induced conditions. Conventional hair care products have attempted to address these concerns through various approaches, including topical application of active ingredients intended to stimulate follicles and improve scalp health. Unfortunately, these attempts have not been entirely or even partially successful.

### GENERAL DESCRIPTION

A hair treatment composition can include one or more delivery systems to improve the stability and targeted delivery of bioactive materials to the hair and scalp. The delivery systems can include lipid vesicles, liposomes, and/or ceramide complexes that encapsulate the same or different bioactive materials. By encapsulating these materials, the compositions can protect them from degradation and more effectively address issues such as hair loss and thinning.

A hair treatment composition can include a first fraction of lipid vesicles encapsulating a first material and a second fraction of lipid vesicles encapsulating a second, different material. The lipid vesicles in one or both fractions can be plant-derived extracellular vesicles, such as exosomes. The first material can be a stimulant material, such as a polyphenol including caffeine and/or epigallocatechin gallate (EGCG), while the second material can be a botanical bioactive material, such as one or more Ayurvedic botanical extracts.

In some configurations, a hair treatment composition can include a base material and lipid vesicles that encapsulate a botanical bioactive material. The base material can also include a non-encapsulated stimulant material, such as caffeine and/or a green tea catechin, dissolved directly within it. This approach allows for a split-release effect, providing both an immediate topical impact from the non-encapsulated stimulant and a sustained, targeted delivery of the encapsulated botanical material.

A hair treatment composition can also be formulated to include three distinct carrier systems. For example, a single composition can include lipid vesicles encapsulating a first bioactive material, liposomes encapsulating a second bioactive material, and ceramide complexes encapsulating a third bioactive material. Each carrier system can be selected to deliver a different type of bioactive material, such as various botanical materials including curcumin and/or Ayurvedic extracts.

The hair treatment compositions can be formulated into various product forms, such as a daily serum, a booster mask, a shampoo, a conditioner, and/or a leave-in composition. These individual products can be designed to work together synergistically as part of a multi-product hair care system. For example, a booster mask can be formulated to provide prolonged follicular availability of its encapsulated botanical bioactives for at least 8 hours following application. A shampoo can be formulated with a sulfate-free surfactant system and a pH of approximately 4.0 to 6.0 to gently cleanse the scalp. In some configurations, the shampoo base comprises a rice water extract infused with one or more amino acids to prepare the scalp and hair for subsequent treatments.

In one embodiment of the hair care system, the rinse-off products (shampoo and conditioner) are formulated without encapsulated materials. The shampoo prepares and revitalizes the scalp and hair using a rice water and amino acid base, while the conditioner maintains and extends the effects of bioactives from leave-on products using non-encapsulated bioactives dissolved directly in the base material. The leave-on products (serum and mask) use the encapsulated delivery systems, with the mask specifically containing only botanical-encapsulating vesicles (second fraction of lipid vesicles) without stimulant-encapsulating vesicles (first fraction of lipid vesicles).

A method of manufacturing a hair treatment composition includes preparing a base material and then cooling it to a temperature of no more than approximately 45° C. to form a cooled base material. The heat-sensitive lipid vesicles and/or other delivery systems are then added to the cooled base material, for example, under low-shear mixing conditions. This process helps to preserve the structural integrity of the delivery systems. The final hair treatment composition can be packaged in an airless container to protect the stability and bioactivity of the encapsulated materials.

The hair treatment compositions can include multiple fractions of lipid vesicles for delivering different bioactive materials to the hair and scalp. The compositions can include a first fraction of lipid vesicles encapsulating a first material and a second fraction of lipid vesicles encapsulating a second material that is different from the first material. The lipid vesicles can be plant-derived extracellular vesicles. The lipid vesicles can be exosomes. The lipid vesicles can have a modal particle size of approximately 30 nm to 300 nm. The first material can be a stimulant material. The second material can be a botanical bioactive material. The composition can include approximately 0.05 to approximately 15 wt % of the first fraction of lipid vesicles and/or approximately 0.05 to approximately 15 wt % of the second fraction of lipid vesicles. The total concentration of lipid vesicles in the composition can be approximately 0.1 to approximately 20 wt %.

The stimulant material can be a polyphenol. The stimulant material can include caffeine. The stimulant material can include one or more green tea catechins such as epigallocatechin gallate (EGCG). The botanical bioactive material can be an Ayurvedic botanical bioactive material. The botanical bioactive material can include one or more extracts selected from Withania somnifera (Ashwagandha), Eclipta prostrata (Bhringraj), Phyllanthus emblica (Amla), Bacopa monnieri (Brahmi), Hibiscus rosa-sinensis, Serenoa repens (Saw Palmetto), and/or Curcuma longa (Curcumin). The lipid vesicles in the first fraction can be derived from the same plant source as the lipid vesicles in the second fraction. The lipid vesicles in the first fraction can be derived from a different plant source than the lipid vesicles in the second fraction.

The hair treatment compositions can include a hybrid delivery system combining encapsulated and non-encapsulated bioactive materials. The composition can include a base material and lipid vesicles encapsulating a botanical bioactive material, wherein the base material includes a non-encapsulated stimulant material. The non-encapsulated stimulant material can be dissolved directly in the aqueous phase of the base material. The composition can include approximately 0.025 to approximately 10 wt % of the lipid vesicles. The composition can include approximately 0.025 to approximately 2 wt % of the non-encapsulated stimulant material. The non-encapsulated stimulant material can include caffeine and/or green tea catechins.

The hair treatment compositions can use multiple distinct carrier systems for delivering different bioactive materials. The composition can include lipid vesicles encapsulating a first bioactive material, liposomes encapsulating a second bioactive material, and ceramide complexes encapsulating a third bioactive material. The first bioactive material can be a stimulant material and/or a botanical bioactive material. The second bioactive material can be curcumin. The third bioactive material can be an Ayurvedic botanical bioactive material. Each carrier system can be present at a concentration of approximately 0.2 wt % to approximately 7 wt %.

The hair treatment compositions can be formulated as various product types within a comprehensive hair care system. The compositions can be formulated as a daily serum, a booster mask, a shampoo, a conditioner, and/or a leave-in composition. A hair care system can include at least three of these product types, wherein each product contains lipid vesicles encapsulating bioactive materials. The booster mask can be formulated to provide prolonged follicular availability of the botanical bioactive material for at least 8 hours following topical application. The booster mask can include approximately 0.025 to approximately 5 wt % of plant-derived extracellular vesicles. The booster mask can include film-forming agents and/or occlusive emollients to create a reservoir on the scalp that extends the availability of the encapsulated bioactives.

A hair shampoo composition can be formulated to prepare and revitalize the scalp for subsequent treatments. The shampoo can comprise rice water extract. The shampoo can comprise one or more amino acids. The shampoo can comprise a nutrient-rich carrier medium, such as a rice water extract, and one or more amino acids. The rice water extract can include inositol. The rice water extract can include vitamins. The rice water extract can include peptides. The rice water extract can include minerals. The materials in the rice water extract can facilitate the deposition and penetration of the amino acids into the hair shaft and follicle. The amino acids, which can include cysteine, methionine, arginine, and lysine, can be selected to mimic the natural keratin profile of human hair. The synergistic activity of the rice water and amino acids can provide both structural reinforcement to the hair shaft and metabolic support to dermal papilla cells, resulting in enhanced tensile strength, reduced breakage, and improved hair density. The shampoo can include a sulfate-free surfactant system. The surfactant system can include sodium cocoyl glutamate, coco-glucoside, babassuamidopropyl betaine, and/or decyl glucoside. The shampoo can have a pH of approximately 4.0 to 6.0.

The manufacturing process for the hair treatment compositions can be designed to preserve the integrity of the heat-sensitive delivery systems. The method can include preparing a base material, cooling the base material to a temperature of no more than approximately 45° C. to form a cooled base material, and adding lipid vesicles to the cooled base material. The lipid vesicles can be added under low-shear mixing conditions. The pH of the composition can be adjusted to approximately 4.5 to approximately 5.5. The composition can be packaged in an airless container to maintain stability and bioactivity. The packaging process can be conducted under a nitrogen blanket to minimize oxygen exposure.

The general description is provided to give a general introduction to the described subject matter as well as a synopsis of some of the technological improvements and/or advantages it provides. The general description and background are not intended to identify essential aspects of the described subject matter, nor should they be used to constrict or limit the scope of the claims. For example, the scope of the claims should not be limited based on whether the recited subject matter includes any or all aspects noted in the general description and/or addresses any of the issues noted in the background.

### DETAILED DESCRIPTION OF EMBODIMENTS

Hair treatment compositions are described that use delivery systems to enhance the bioavailability and efficacy of bioactive materials for addressing hair loss, thinning, and overall scalp health. The compositions can include multiple approaches to encapsulation and delivery, providing both immediate and sustained release of active ingredients.

The hair treatment compositions can include dual fractions of lipid vesicles for targeted delivery of different bioactive materials. A first fraction of lipid vesicles can encapsulate a first material while a second fraction of lipid vesicles can encapsulate a second, different material. This dual-fraction approach allows for the simultaneous delivery of multiple active ingredients with potentially different release profiles and mechanisms of action.

The lipid vesicles can be plant-derived extracellular vesicles, and more specifically can be exosomes. Exosomes are naturally occurring lipid vesicles that can range in size from approximately 30 nanometers to 300 nanometers in modal particle size. These plant-derived extracellular vesicles can be obtained from various botanical sources such as Eclipta prostrata (Bhringraj), Withania somnifera (Ashwagandha), Rosa damascena (Rose), Aloe barbadensis, and/or other plant sources. The use of plant-derived extracellular vesicles provides a natural, biocompatible delivery system that can effectively penetrate the scalp and deliver bioactive materials to hair follicles.

The first material encapsulated within the first fraction of lipid vesicles can be a stimulant material. The stimulant material can include polyphenolic compounds such as caffeine and/or green tea catechins. Green tea catechins can include epigallocatechin gallate (EGCG), which can protect hair cells and stimulate follicles. The stimulant material can help improve blood circulation to the scalp, energize hair follicles, and potentially inhibit hormones associated with hair loss.

The second material encapsulated within the second fraction of lipid vesicles can be a botanical bioactive material. The botanical bioactive material can include one or more Ayurvedic botanical extracts selected from traditional medicinal plants. These can include extracts from Withania somnifera (Ashwagandha), Eclipta prostrata (Bhringraj), Phyllanthus emblica (Amla), Bacopa monnieri (Brahmi), Hibiscus rosa-sinensis, Serenoa repens (Saw Palmetto), Curcuma longa (Curcumin), and/or combinations thereof. Each of these botanical materials can provide specific benefits for hair health, such as reducing stress-induced hair loss, strengthening hair follicles, preventing premature graying, and/or promoting overall scalp health.

The concentration of lipid vesicles in the hair treatment compositions can vary depending on the specific formulation and intended use. The first fraction of lipid vesicles can be present at a concentration of approximately 0.05 wt % to approximately 15 wt % of the total composition (or 1-10 wt % or 2-5 wt %). The second fraction of lipid vesicles can also be present at a concentration of approximately 0.05 wt % to approximately 15 wt % (or 1-10 wt % or 2-5 wt %). The total concentration of lipid vesicles (combining both fractions) can range from approximately 0.1 wt % to approximately 20 wt % of the composition (or 2-15 wt % or 4-10 wt %).

In specific formulations, such as a daily hair growth serum, the first fraction of lipid vesicles encapsulating stimulant materials can be present at approximately 3 wt %, while the second fraction encapsulating botanical bioactives can be present at approximately 4 wt %, providing a total lipid vesicle concentration of approximately 7 wt %.

The hair treatment compositions can also employ a hybrid delivery strategy that combines encapsulated and non-encapsulated bioactive materials. In such formulations, the composition can include a base material containing non-encapsulated stimulant material dissolved directly in the aqueous phase, along with lipid vesicles that encapsulate botanical bioactive materials. This approach provides both immediate topical effects from the non-encapsulated stimulant and sustained, targeted delivery from the encapsulated botanicals.

The non-encapsulated stimulant material can include caffeine at a concentration of approximately 0.30 to 0.40 wt % (or 0.05-2 wt %) and/or EGCG at approximately 0.10 to 0.15 wt % (or 0.025-1 wt %) dissolved directly in the base material. The combination of free and encapsulated actives creates a split-release system that can provide both immediate stimulation of hair follicles and long-term nourishment from the protected botanical ingredients.

The hair treatment compositions can use multiple distinct carrier systems to deliver different types of bioactive materials. A single composition can include lipid vesicles encapsulating a first bioactive material, liposomes encapsulating a second bioactive material, and ceramide complexes encapsulating a third bioactive material. This multi-carrier approach allows for the optimization of delivery of different types of active ingredients based on their chemical properties and desired release profiles.

The liposomes can encapsulate materials such as curcumin, which can provide anti-inflammatory and antioxidant benefits. The ceramide complexes can encapsulate Ayurvedic botanical materials such as Ashwagandha extract, providing strengthening and barrier-reinforcing properties to the hair and scalp. Each carrier system can be present at concentrations ranging from approximately 0.2 wt % to approximately 7 wt % (or 0.1-10 wt %), depending on the specific formulation requirements.

The hair treatment compositions can be formulated into various product types to address different hair care needs and usage patterns. These product forms can include daily serums, booster masks, shampoos, conditioners, and leave-in treatments. Each product type can be specifically formulated to work synergistically within a comprehensive hair care system.

A daily serum can be formulated as a lightweight, water-based composition suitable for direct scalp application. The serum can include dual fractions of lipid vesicles as described above, along with supporting ingredients such as glycerin, niacinamide, panthenol, sodium hyaluronate, and natural preservatives. The serum can be packaged in an airless applicator pen designed to dispense metered doses of approximately 0.08 cubic centimeters per actuation, ensuring consistent and hygienic application.

A booster mask can be formulated as a rich, creamy oil-in-water emulsion designed for periodic intensive treatment. The mask can include lower concentrations of lipid vesicles, such as approximately 0.025 wt % to approximately 5 wt % (or 1-3 wt %), but can be formulated with film-forming agents and occlusive emollients to create a reservoir effect on the scalp. This reservoir can provide prolonged follicular availability of the encapsulated botanical bioactives for at least 8 hours and potentially up to 36 hours following application and rinsing.

The mask formulation can include a water phase containing humectants and chelating agents, and an oil phase containing fatty alcohols such as cetearyl alcohol and stearyl alcohol, cationic conditioning agents such as behentrimonium methosulfate, and nourishing emollients such as shea butter and coconut oil. Film-forming agents such as hydrolyzed quinoa protein and/or hydrogenated lecithin can be incorporated to enhance the substantivity of the treatment on the scalp and hair.

Shampoo formulations can be designed to gently cleanse while delivering beneficial nutrients to the hair and scalp, preparing them for leave-in treatments. The shampoo can be based on a nutrient-rich carrier medium, such as a rice water extract containing naturally occurring inositol, vitamins, peptides, and minerals. This base can be infused with one or more amino acids, for example, at a concentration between approximately 0.01 to 5 wt % (or 0.5 to 4 wt % or 1 to 3 wt %). The amino acids can be selected from cysteine, methionine, arginine, lysine, serine, glycine, proline, alanine, and/or glutamic acid.

The amino acids can be chosen to mimic the natural keratin amino acid profile of human hair. The synergistic combination of the rice water extract's inositol and the exogenously added amino acids provides (a) structural reinforcement of keratin proteins in the hair shaft and/or (b) metabolic support to dermal papilla cells within the follicle. This activity enhances the tensile strength of hair fibers, reduces breakage, improves elasticity, and helps prolong the anagen phase of the hair growth cycle. The formulation is designed to produce both immediate cosmetic benefits, such as enhanced luster and smoothness, and long-term therapeutic benefits, including improved hair density, follicle vitality, and reduced shedding. The shampoo can include a sulfate-free surfactant system and be formulated with a pH of approximately 4.0 to approximately 6.0. Suitable surfactants can include sodium cocoyl glutamate, coco-glucoside, babassuamidopropyl betaine, and/or decyl glucoside.

Conditioner formulations can be designed to maintain and extend the effects of bioactives delivered by other products in the hair care system, such as the daily serum. The conditioner can contain fatty alcohols and cationic conditioning agents for detangling and smoothing, along with plant-based oils for nourishment. The pH can be adjusted to approximately 4.0 to 5.0 to close the hair cuticle and enhance the deposition of conditioning agents. The conditioner can include bioactives (e.g., any one or combination of the bioactives described in this document), such as botanical extracts or peptides, dissolved or dispersed directly within its base, rather than in encapsulated form.

Leave-in treatments can be formulated as lightweight sprays, lotions, or creams that provide ongoing protection and nourishment throughout the day. These formulations can include thermal and UV protection agents, along with lipid vesicles delivering botanical bioactives. The leave-in products can also include film-forming polymers such as VP/VA copolymer to provide light hold and style memory while creating a protective barrier on the hair.

The manufacturing process for these hair treatment compositions can include control of temperature and mixing conditions to preserve the integrity of the heat-sensitive delivery systems. The general manufacturing method includes preparing a base material containing water-soluble ingredients and any heat-stable components. This base material is then cooled to a temperature of no more than approximately 45° C., and preferably to approximately 40° C. or below.

Once the base material has reached the appropriate temperature, the lipid vesicles and other heat-sensitive encapsulation systems are added under low-shear mixing conditions. Low-shear mixing, such as using a paddle mixer at low revolutions per minute, helps preserve the structural integrity of the vesicles while ensuring uniform distribution throughout the composition. The mixing should be sufficient to achieve homogeneity without causing excessive aeration or mechanical stress on the delivery systems.

Following the incorporation of the encapsulated materials, the pH of the composition can be adjusted to fall within the target range of approximately 4.5 to approximately 5.5 using citric acid or other suitable pH adjusters. This pH range is compatible with the scalp and helps maintain the stability of both the encapsulation systems and the bioactive materials.

The finished compositions can be packaged in airless containers to minimize oxidation and maintain the stability of the encapsulated bioactives. The packaging process can be conducted under a nitrogen blanket to further reduce oxygen exposure. Airless packaging systems, such as airless pumps, tubes with one-way valves, or refillable cartridge systems, help preserve the bioactivity of the formulations throughout their shelf life.

Quality control measures can include particle size analysis of the lipid vesicles using techniques such as Nanoparticle Tracking Analysis (NTA) to ensure the modal particle size remains within the target range of 30 to 300 nanometers. The bioactivity of encapsulated materials can be monitored using High-Performance Liquid Chromatography (HPLC) or other appropriate analytical methods.

The hair treatment compositions can be designed to work as part of a comprehensive hair care system. The daily serum provides consistent delivery of both stimulant and botanical bioactives for ongoing follicular support. The booster mask offers intensive periodic treatment to replenish the scalp's reservoir of bioactive materials. The shampoo and conditioner maintain scalp health and provide additional bioactive delivery during the cleansing and conditioning routine. The leave-in treatment offers protection and continued nourishment throughout the day.

This systematic approach allows users to address hair loss and thinning through multiple mechanisms of action, including DHT inhibition, improved circulation, stress reduction, follicle strengthening, and scalp health maintenance. The use of encapsulation technologies ensures that sensitive bioactive materials are protected from degradation and can be effectively delivered to their target sites of action.

The flexibility of the formulation platform allows for customization based on specific hair and scalp needs. Different combinations of encapsulated and non-encapsulated actives can be selected to address various causes of hair loss, from hormonal factors to stress-induced shedding. The concentration of lipid vesicles and other delivery systems can be adjusted to optimize efficacy while maintaining cosmetic elegance and user acceptability.

The hair treatment compositions represent an advancement in the delivery of natural bioactive materials for hair care applications. By combining traditional Ayurvedic botanicals with modern encapsulation technologies, these formulations can provide enhanced bioavailability and sustained delivery of beneficial ingredients to support hair growth and scalp health.

### EXAMPLES

The following examples are provided to further illustrate the disclosed subject matter. They should not be used to constrict or limit the scope of the claims in any way.

### Example 1—Hair Treatment Serum with Dual Fractions of Encapsulated Bioactives

A hair treatment serum is prepared comprising two distinct fractions of lipid vesicles. The serum is formulated as a water-based, leave-in composition suitable for daily scalp application. The first fraction of lipid vesicles is present at approximately 3.0 wt % of the total composition and encapsulates a stimulant material. The stimulant material comprises a blend of polyphenols, which can include caffeine and/or green tea catechins such as epigallocatechin gallate (EGCG). The second fraction of lipid vesicles is present at approximately 4.0 wt % of the total composition and encapsulates a botanical bioactive material. The botanical bioactive material can comprise one or more Ayurvedic botanical extracts selected from the group of Withania somnifera (Ashwagandha), Eclipta prostrata (Bhringraj), Phyllanthus emblica (Amla), Bacopa monnieri (Brahmi), Hibiscus rosa-sinensis, Serenoa repens (Saw Palmetto), and/or Curcuma longa (Curcumin). The total concentration of lipid vesicles in the final composition is approximately 7.0 wt %. Both fractions of lipid vesicles are plant-derived extracellular vesicles, also referred to as exosomes, with a modal particle size of approximately 30 nm to 300 nm. The serum base includes water, glycerin, niacinamide, panthenol, sodium hyaluronate, a preservative system, and a pH adjuster.

The serum is manufactured by first preparing an aqueous base that contains the water-soluble components. The base is then cooled to a temperature of approximately 40° C. Following this cooling step, the first fraction of lipid vesicles and the second fraction of lipid vesicles are added to the cooled base under conditions of low-shear mixing to preserve the structural integrity of the vesicles. The pH of the final composition is adjusted to a range of 4.8 to 5.3. The finished serum is packaged in an airless applicator pen, which is designed to dispense a metered dose of approximately 0.08 cc per actuation.

### Example 2—Hair Treatment Serum with Encapsulated Botanicals and Non-Encapsulated Stimulant

A hybrid hair treatment serum is prepared containing a single fraction of lipid vesicles and a non-encapsulated stimulant material dissolved directly in the base of the composition. The composition includes a fraction of plant-derived lipid vesicles (exosomes) at a concentration of approximately 4.0 wt %. These lipid vesicles encapsulate an Ayurvedic botanical bioactive material that can comprise one or more extracts selected from the group of Withania somnifera (Ashwagandha), Eclipta prostrata (Bhringraj), Phyllanthus emblica (Amla), and/or Bacopa monnieri (Brahmi). The aqueous base of the serum contains a non-encapsulated stimulant material, including approximately 0.30 wt % to 0.40 wt % of caffeine and approximately 0.10 wt % to 0.15 wt % of epigallocatechin gallate (EGCG), which are fully dissolved in the water phase.

The serum is manufactured by dissolving the caffeine and EGCG in the aqueous phase of the base material, which can be heated moderately to ensure complete dissolution. The base is subsequently cooled to a temperature of no more than 45° C. The single fraction of lipid vesicles encapsulating the botanical bioactive material is then added to the cooled base material with gentle agitation. The final pH is adjusted to approximately 5.0, and the serum is packaged in an airless container.

### Example 3—Hair Treatment Serum with Split-Release Stimulant Material

A hair treatment serum is formulated to provide a split release of a stimulant material, having both an immediate-release and a sustained-release component. The composition comprises a first fraction of lipid vesicles (Exo-1) present at 1.5 wt %, which encapsulates a stimulant material such as caffeine. A second fraction of lipid vesicles (Exo-2) is included at 4.0 wt % and encapsulates an Ayurvedic botanical bioactive material. In addition to the encapsulated stimulant, the aqueous base of the serum contains approximately 0.15 wt % to 0.25 wt % of non-encapsulated, or “free,” caffeine. The total concentration of lipid vesicles in the final composition is approximately 5.5 wt %.

The manufacturing process for this serum involves dissolving the non-encapsulated caffeine in the water phase of the base material. The base is then cooled to a temperature of approximately 40° C. to 45° C. In this cooling phase, both the first fraction of lipid vesicles (Exo-1) and the second fraction of lipid vesicles (Exo-2) are incorporated into the composition under low-shear mixing conditions. The final serum is adjusted to a pH of approximately 4.9 and is packaged in a metered-dose airless applicator.

### Example 4—Hair Treatment Serum with Botanical Exosomes and Non-Exosomal Encapsulated Stimulant

A hair treatment serum is prepared that uses two different types of nanoparticle delivery systems for different active materials. The formulation includes a fraction of plant-derived exosomes at a concentration of approximately 3.0 wt % to 5.0 wt %. These exosomes exclusively encapsulate an Ayurvedic botanical bioactive material. The composition also contains a separate dispersion of nano-encapsulated caffeine at a concentration of approximately 0.20 wt % to 0.60 wt %. The caffeine in this system is encapsulated within polymeric nanoparticles, which are structurally distinct from the exosomes. No stimulant material is encapsulated within the exosome fraction. The aqueous base further comprises humectants, preservatives, and supporting ingredients such as niacinamide.

To manufacture this serum, a base material is prepared and subsequently cooled to a temperature below 45° C. The suspension of exosomes encapsulating the botanical material and the separate dispersion of nano-encapsulated caffeine are both added to the cooled base material. The components are gently mixed to achieve a homogeneous final composition. The pH is adjusted to a scalp-compatible range of 4.5 to 5.5, and the product is filled into airless packaging.

### Example 5—Hair Treatment Serum with Stimulant Liposomes and Botanical Exosomes

A hair treatment serum is formulated using a combination of liposomes and exosomes as distinct delivery vehicles for different active materials. The composition includes a first encapsulated fraction comprising liposomes at a concentration of approximately 0.2 wt % to 2.0 wt %. These liposomes encapsulate a stimulant material, such as caffeine. The composition also includes a second encapsulated fraction comprising plant-derived exosomes at a concentration of approximately 2.0 wt % to 7.0 wt %. These exosomes encapsulate an Ayurvedic botanical bioactive material. The serum base is formulated to be free of non-encapsulated caffeine.

The manufacturing method for this serum involves the preparation of a stable aqueous base, which is then cooled to a temperature of approximately 40° C. During this cooling phase, a previously prepared liposome dispersion containing the stimulant material and an exosome suspension containing the botanical bioactive material are added to the base. The mixture is stirred gently to ensure uniform distribution of both carrier systems without disrupting their respective structures. The final serum is packaged in an airless dispenser.

### Example 6—Hair Treatment Serum with Three Distinct Encapsulation Systems

A multi-carrier hair treatment serum is prepared, incorporating three different types of encapsulation systems for the targeted delivery of distinct bioactive materials. The first system comprises plant-derived exosomes at a concentration of 2.0 wt % to 6.0 wt %, which encapsulate a first Ayurvedic botanical bioactive material. The second system comprises liposomes at a concentration of 0.5 wt % to 3.0 wt %; these liposomes encapsulate curcumin, yielding a final curcumin concentration of approximately 0.1 wt % to 1.0 wt % in the total composition. The third system comprises ceramide complexes at a concentration of 0.2 wt % to 2.0 wt %, which encapsulate a second Ayurvedic botanical bioactive material, such as Withania somnifera (Ashwagandha) extract.

This serum is manufactured by preparing the aqueous base and cooling it to a temperature of no more than 45° C. The three distinct carrier systems—the exosome suspension, the liposome dispersion, and the ceramide complexes—are then added sequentially to the cooled base under low-shear mixing conditions to form a stable and homogeneous final composition. The pH is adjusted to approximately 5.2, and the product is packaged in an airless container.

### Example 7—High-Concentration Dual-Exosome Hair Treatment Serum

A high-concentration hair treatment serum is formulated with two distinct fractions of lipid vesicles. The composition includes a first fraction of plant-derived exosomes at approximately 5.0 wt %, which encapsulates a stimulant material comprising caffeine and/or green tea catechins. The composition also includes a second fraction of plant-derived exosomes at approximately 7.0 wt %, which encapsulates an Ayurvedic botanical bioactive material. The total concentration of lipid vesicles in the final formulation is approximately 12.0 wt %, which is within a broader possible range of 0.1 wt % to 20 wt %. The aqueous base is formulated with a suitable rheology modifier to accommodate the higher solids content from the vesicles and to maintain a desirable viscosity for application.

The serum is produced by preparing the aqueous base and cooling it to 40° C. The two separate, concentrated exosome fractions are then added to the cooled base under controlled, low-shear mixing. The final pH is adjusted to 5.1, and the resulting high-concentration serum is packaged in a dose-controlled airless applicator.

### Example 8—Hair Treatment Serum Utilizing Specific Plant-Derived Extracellular Vesicles

A series of hair treatment serums is prepared based on the formulation described in Example 1 to demonstrate the use of extracellular vesicles from various plant sources. In a first preparation, the lipid vesicles for both the stimulant-encapsulating fraction and the botanical-encapsulating fraction are derived from a single plant source, such as Eclipta prostrata (Bhringraj), Withania somnifera (Ashwagandha), Rosa damascena (Rose), and/or Aloe barbadensis. In another preparation, a composition is formulated using vesicles from two different sources, where the first fraction of vesicles (encapsulating the stimulant) is derived from Rosa damascena and the second fraction of vesicles (encapsulating the botanical material) is derived from Eclipta prostrata. In all preparations, the vesicles exhibit a modal particle size in the range of 30 nm to 300 nm, as can be determined by Nanoparticle Tracking Analysis (NTA), and are added to the serum base during the cooling phase of manufacture.

### Example 9—Packaging and Stability Assessment of a Hair Treatment Serum

The stability of the hair treatment serum composition prepared according to Example 1 is assessed. The final serum, comprising 3.0 wt % of a first exosome fraction and 4.0 wt % of a second exosome fraction, is packaged into an airless applicator pen with a replaceable cartridge system. The filling process is conducted under a nitrogen blanket to ensure that the oxygen content in the container headspace is below 2% by volume. The packaged samples are then subjected to a stability study by storing them in a controlled environmental chamber at 25° C. and 60% relative humidity for a period of three months.

At the conclusion of the storage period, the samples are analyzed. The bioactivity of the encapsulated materials is measured, for example, via High-Performance Liquid Chromatography (HPLC), and is retained at a level of at least 85% of the initial value. The physical stability of the vesicles is evaluated, for example, using Nanoparticle Tracking Analysis (NTA), which confirms that the modal particle size of the vesicles in both fractions remains within +20% of the initial measurement. These results indicate good physical and chemical stability of the delivery systems within the formulation when protected by the specified airless packaging.

### Example 10—Low-Concentration Botanical Exosome Booster Mask (>8 Hour Availability)

A hair treatment mask is prepared as a rich, creamy emulsion designed to function as a booster composition. The mask is formulated to be applied to the scalp and hair for a short duration (e.g., 5-15 minutes) before rinsing, leaving behind a conditioning film that provides prolonged follicular availability of its active ingredients. The composition includes a single fraction of plant-derived lipid vesicles (exosomes) at a very low concentration of approximately 0.12 wt %. These exosomes encapsulate an Ayurvedic botanical bioactive material. The botanical bioactive material can comprise one or more extracts selected from the group of Withania somnifera (Ashwagandha), Eclipta prostrata (Bhringraj), Phyllanthus emblica (Amla), Bacopa monnieri (Brahmi), and/or Serenoa repens (Saw Palmetto).

The base of the mask is an oil-in-water emulsion comprising a water phase and an oil phase. The water phase includes water, a humectant such as glycerin, and a chelating agent such as sodium phytate. The oil phase includes a blend of fatty alcohols such as cetearyl alcohol and stearyl alcohol to provide structure and viscosity; a cationic conditioning agent such as Behentrimonium Methosulfate for detangling and softness; and a selection of emollients such as Butyrospermum Parkii (Shea) Butter and Cocos nucifera (Coconut) Oil to nourish the hair and form a substantive film on the scalp. The formulation also includes a film-forming agent, such as hydrolyzed quinoa protein, which can couple with the emollients to create a breathable matrix on the scalp.

The mask is manufactured by preparing the water phase (Phase A) and the oil phase (Phase B) in separate vessels. Phase A is heated to approximately 75° C. Phase B, containing the fatty alcohols, conditioning agent, and emollients, is heated in a separate vessel to approximately 75° C. until all components are melted and homogeneous. Phase B is then slowly added to Phase A under high-shear homogenization to form a stable emulsion. The emulsion is then allowed to cool with gentle, continuous stirring. When the temperature of the emulsion reaches approximately 40° C. or below, the exosome suspension, the hydrolyzed quinoa protein, a preservative system, and a fragrance are added. The final pH of the mask is adjusted to a range of 4.5 to 5.5. The formulation is designed such that, after rinsing, the residual film on the scalp provides a reservoir of exosomes, enabling the sustained release and follicular availability of the encapsulated botanical bioactives for a period of at least 8 hours.

### Example 11—Minimum-Threshold Botanical Exosome Booster Mask (>8 Hour Availability)

A hair treatment booster mask is prepared with a composition analogous to that of Example 10, but with the concentration of the botanical-encapsulating exosomes adjusted to a minimum threshold of approximately 0.10 wt %. This formulation is designed to demonstrate the booster effect at a very low level of active inclusion. The exosomes encapsulate an Ayurvedic botanical bioactive material which can comprise one or more extracts selected from the group of Withania somnifera (Ashwagandha), Eclipta prostrata (Bhringraj), Phyllanthus emblica (Amla), and/or Bacopa monnieri (Brahmi).

The mask base is a conditioning cream comprising water, Aloe Barbadensis Leaf Juice, cetearyl alcohol, stearamidopropyl dimethylamine as a cationic conditioner, and a blend of emollients such as Helianthus Annuus (Sunflower) seed oil and Squalane. To enhance the prolonged availability effect, the formulation includes approximately 0.5 wt % of hydrogenated lecithin, which can form a substantive lipid layer on the scalp that helps to retain the exosomes at the follicular opening.

The manufacturing process follows an oil-in-water emulsion procedure. The water-phase ingredients are combined and heated to 70° C. The oil-phase ingredients, including the fatty alcohol, conditioner, oils, and squalane, are combined and heated to 70° C. The two phases are emulsified using a homogenizer. The resulting emulsion is cooled under gentle agitation. At a temperature of 45° C. or below, the exosome suspension (at 0.10 wt %), the hydrogenated lecithin, preservatives, and fragrance are incorporated into the batch. The final pH is adjusted to approximately 5.0. This composition is designed to provide prolonged follicular availability of the encapsulated botanicals for at least 8 hours following application and rinsing.

### Example 12—High-Concentration Botanical Exosome Booster Mask (8-36 Hour Availability)

A high-concentration hair treatment booster mask is prepared. This formulation is designed to create a significant reservoir of bioactives on the scalp for extended, multi-day follicular support. The composition includes a single fraction of plant-derived exosomes at a concentration of approximately 4.0 wt %. These exosomes encapsulate an Ayurvedic botanical bioactive material. The botanical bioactive material can comprise one or more extracts selected from the group of Withania somnifera (Ashwagandha), Eclipta prostrata (Bhringraj), Phyllanthus emblica (Amla), Bacopa monnieri (Brahmi), and/or Serenoa repens (Saw Palmetto).

The mask base is specifically formulated to support the higher solids load from the exosomes and to maximize the duration of the booster effect. It is a rich, dense cream emulsion. The oil phase comprises a higher level of structural lipids and waxes, such as Cetearyl Alcohol and Euphorbia Cerifera (Candelilla) Wax, in combination with Behentrimonium Methosulfate. The stabilizer system in the water phase is reinforced with a combination of Xanthan Gum and Sclerotium Gum to ensure long-term emulsion stability. The base also includes film-forming polysaccharides and occlusive emollients such as Shea Butter to create a durable, flexible film on the scalp.

The mask is manufactured by creating a stable oil-in-water emulsion as described in the previous examples, with heating of the phases to 75° C.-80° C. After emulsification, the batch is cooled with moderate agitation. Due to the higher concentration of vesicles, the exosome suspension is added to the cooled base (<45° C.) very slowly and with controlled mixing to provide uniform dispersion without causing instability. The final pH is adjusted to 4.5-5.5. This high-concentration formulation is designed to extend the follicular availability of the encapsulated botanical bioactives for a period of up to 36 hours after application, providing a potent weekly or bi-weekly treatment that complements a daily serum.

### Example 13—Dual-Exosome Fraction Booster Mask

A hair treatment booster mask is prepared that contains two distinct fractions of plant-derived exosomes, mirroring a dual-exosome daily serum formulation. The first fraction of lipid vesicles (Exo-1) is included at a concentration of approximately 0.2 wt % to 1.0 wt % and encapsulates a stimulant material, such as caffeine and/or green tea catechins. The second fraction of lipid vesicles (Exo-2) is included at a higher concentration of approximately 0.5 wt % to 4.0 wt % and encapsulates an Ayurvedic botanical bioactive material. In an alternative embodiment, the first fraction of lipid vesicles (Exo-1) is absent, and the mask contains only the botanical-encapsulating exosome fraction (Exo-2) to specifically reinforce the botanical activity of a corresponding serum.

The mask base is a conditioning emulsion similar to that described in Example 10. The manufacturing process involves preparing the emulsion base, cooling it to a temperature of 40° C., and then incorporating the one or two distinct exosome suspensions into the cooled base. If two fractions are used, they can be added sequentially or as a pre-blended mixture. This formulation allows the mask to function as a comprehensive booster for a multi-pathway hair care system, with a particular emphasis on replenishing the scalp's reservoir of the botanical bioactives from the more concentrated Exo-2 fraction.

### Example 14—Triple-Carrier System Booster Mask

A hair treatment booster mask is prepared utilizing three separate and distinct bioactive delivery systems. The first system comprises plant-derived exosomes at a concentration of approximately 0.25 wt % to 2.0 wt %; these exosomes encapsulate a first botanical bioactive material, such as any of those described above. The second system comprises liposomes at a concentration of approximately 0.5 wt % to 2.5 wt %; these liposomes encapsulate curcumin. The third system comprises ceramide complexes at a concentration of approximately 0.3 wt % to 1.5 wt %; these complexes encapsulate a second botanical bioactive material such as any of those described above.

The mask is manufactured by first creating a stable oil-in-water emulsion base with conditioning agents and emollients. The base is prepared and cooled to a temperature of no more than 45° C. During this final cooling phase, the three pre-formed, distinct carrier dispersions are incorporated into the batch. The exosome suspension, the liposome dispersion, and the ceramide complexes are added sequentially to the base under conditions of low-shear mixing to ensure their uniform distribution and to maintain the structural integrity of each unique carrier. The final composition is designed to provide a multi-faceted booster treatment, delivering different bioactives via different encapsulation technologies to potentially target different pathways related to scalp health and follicular function.

### Example 15—Shampoo with Rice Water Extract and Amino Acid Complex

A hair shampoo composition is prepared to revitalize the scalp and infuse hair fibers with structural components. The formulation is based on a nutrient-rich rice water extract and an amino acid blend, and it is free of exosomes.

TABLE 1
Formulation

| Ingredient (INCI Name) | Function | wt % |
|---|---|---|
| Deionized Water | Solvent | q.s. |
| | | to 100 |
| Saccharomyces/Rice Ferment Filtrate | Nutrient Carrier | 15.00 |
| (Rice Water Extract) | | |
| Sodium Cocoyl Isethionate | Primary Surfactant | 8.00 |
| Lauryl Glucoside | Co-Surfactant | 6.00 |
| Cocamidopropyl Betaine | Co-Surfactant | 4.00 |
| Glycerin | Humectant | 3.00 |
| Amino Acid Blend (Arginine, Alanine, | Hair Repair/ | 1.50 |
| Serine, Valine, Proline, Threonine, | Strengthening | |
| Isoleucine, Histidine, Phenylalanine) | | |
| Guar Hydroxypropyltrimonium Chloride | Conditioning Agent | 0.50 |
| Caprylhydroxamic Acid, Glyceryl | Preservative System | 1.00 |
| Caprylate, Glycerin | | |
| Citric Acid | pH Adjuster | As |
| | | needed |

### Manufacturing Procedure:

In a primary manufacturing vessel, Deionized Water and Rice Water Extract are combined. Under moderate propeller agitation, the surfactants (Sodium Cocoyl Isethionate, Lauryl Glucoside, Cocamidopropyl Betaine) are added and the mixture is gently heated to 45-50° C. to ensure they are fully dissolved and blended. The batch is then allowed to cool. Once the temperature is below 40° C., the Glycerin, Amino Acid Blend, Guar Hydroxypropyltrimonium Chloride, and preservative system are added sequentially with slow mixing. The pH of the final shampoo composition is measured and adjusted with Citric Acid to a target range of 4.5 to 5.5. The resulting product is a gentle, nutrient-rich shampoo designed to improve hair strength and elasticity while preparing the scalp for leave-on treatments.

### Example 16—Surfactant-Compatible Shampoo with Exosomes and Nano-Encapsulated Caffeine

A hair shampoo composition is prepared to demonstrate the stable incorporation of both plant-derived exosomes and nano-encapsulated actives within a mild, sulfate-free cleansing system. The formulation is designed to gently cleanse the scalp and hair while delivering bioactive compounds to support scalp health and hair growth.

TABLE 2
Formulation

| Phase | Ingredient (INCI Name) | Function | wt % |
|---|---|---|---|
| A | Deionized Water | Solvent | q.s. |
| | | | to 100 |
| A | Glycerin | Humectant | 3.00 |
| A | Xanthan Gum | Thickener/Stabilizer | 0.80 |
| B | Sodium Cocoyl Glutamate | Primary Surfactant | 7.00 |
| | | (Anionic) | |
| B | Coco-Glucoside | Co-Surfactant (Non-ionic) | 5.00 |
| B | Babassuamidopropyl | Co-Surfactant (Amphoteric) | 4.00 |
| | Betaine | | |
| B | Decyl Glucoside | Co-Surfactant (Non-ionic) | 4.00 |
| C | Plant-Derived Exosomes | Bioactive Delivery Vehicle | 2.00 |
| | (from Eclipta prostrata) | | |
| C | Nano-Encapsulated | Stimulant | 2.50 |
| | Caffeine | | |
| C | Leuconostoc/Radish Root | Preservative | 2.00 |
| | Ferment Filtrate | | |
| C | Sodium Benzoate | Preservative | 0.50 |
| C | Citric Acid | pH Adjuster | As |
| | | | needed |
| C | Fragrance (Essential | Fragrance | 0.50 |
| | Oil Blend) | | |

The plant-derived exosomes in Phase C encapsulate one or more Ayurvedic botanical bioactive materials, which can comprise one or more extracts selected from the group of Withania somnifera (Ashwagandha), Eclipta prostrata (Bhringraj), Phyllanthus emblica (Amla), and/or Bacopa monnieri (Brahmi).

### Manufacturing Procedure:

Phase A Preparation: In a primary manufacturing vessel, Deionized Water is charged. Under moderate propeller agitation, Glycerin is added and mixed until uniform. Xanthan Gum is then slowly dispersed into the vortex to prevent the formation of agglomerates. The mixture is heated to 75° C. and held for 20 minutes to ensure complete hydration of the gum, forming a smooth, uniform gel.

Phase B Preparation: In a separate vessel, the surfactants of Phase B (Sodium Cocoyl Glutamate, Coco-Glucoside, Babassuamidopropyl Betaine, and Decyl Glucoside) are combined and gently heated to 45-50° C. with slow mixing to create a homogeneous surfactant blend.

Combination: The surfactant blend (Phase B) is slowly added to the hydrated gum phase (Phase A) with continued moderate agitation. Mixing is continued until the entire batch is uniform and clear.

Cooling: The batch is then allowed to cool with slow, continuous mixing to prevent excessive aeration.

Phase C Addition (Cool-Down Phase): When the temperature of the batch reaches 40° C. or below, the components of Phase C are added sequentially. The suspension of plant-derived exosomes is added first, followed by the dispersion of nano-encapsulated caffeine. The preservatives (Leuconostoc/Radish Root Ferment Filtrate and Sodium Benzoate) and fragrance are then added. Mixing is maintained at a low speed throughout this phase to ensure gentle, uniform incorporation of the heat-sensitive active ingredients.

Final Adjustments: The pH of the final shampoo composition is measured and adjusted with a Citric Acid solution to a target range of 4.5 to 5.5. The viscosity is measured to confirm it is within specification.

Final Product Characteristics: The resulting product is a translucent, viscous gel shampoo. The use of a mild, sulfate-free surfactant system is selected to minimize potential disruption to the exosome lipid bilayer, thereby preserving the integrity of the bioactive delivery system within the cleansing formulation.

### Example 17—Shampoo with a Blend of Plant E V Nanovesicles from Multiple Sources

A hair shampoo is prepared to demonstrate the versatility of using a blend of plant-derived extracellular vesicle (EV) nanovesicles from multiple botanical sources. The formulation is designed to provide a broad spectrum of plant-derived bioactives within a gentle, pH-balanced cleansing base.

TABLE 3
Formulation

| Ingredient (INCI Name) | Function | wt % |
|---|---|---|
| Deionized Water | Solvent | q.s. to 100 |
| Disodium Cocoyl Glutamate | Mild Surfactant | 10.00 |
| Lauryl Glucoside | Mild Surfactant | 5.00 |
| Acrylates Copolymer | Thickener | 1.50 |
| Plant EV Nanovesicle Blend (Rosa | Bioactive Delivery | 0.75 |
| damascena + Eclipta prostrata) | Vehicle | |
| Panthenol (Pro-Vitamin B5) | Humectant/Hair Repair | 0.50 |
| Benzyl Alcohol, Salicylic Acid, | Preservative System | 1.00 |
| Glycerin, Sorbic Acid | | |
| Sodium Hydroxide | pH Adjuster | As needed |

The Plant EV Nanovesicle Blend encapsulates one or more Ayurvedic botanical bioactive materials derived from their source plants.

### Manufacturing Procedure:

Base Preparation: In the primary vessel, Acrylates Copolymer is dispersed in Deionized Water. The pH is partially neutralized with Sodium Hydroxide to begin building viscosity. The surfactants are then added and mixed until uniform.

Cooling: The batch is allowed to cool to room temperature (approximately 25° C.).

Active Addition: Once cooled, the Plant EV Nanovesicle Blend is added with very slow, gentle mixing. Panthenol and the preservative system are then incorporated.

Final Adjustments: The pH is adjusted with Sodium Hydroxide to a final target range of 4.0 to 6.0, which also completes the thickening of the Acrylates Copolymer, resulting in the final desired viscosity.

Final Product Characteristics: The final product is a clear, viscous shampoo gel. This example illustrates that a stable and effective bioactive shampoo can be formulated using a cocktail of EVs from different plant origins, broadening the scope of potential active ingredients and their synergistic effects.

### Example 18—Multi-Carrier Hair Conditioner with Exosomes, Nano-Caffeine, and Liposomal Curcumin

A rinse-out hair conditioner is prepared as a rich, creamy emulsion. The formulation is designed to condition, detangle, and smooth the hair shaft while depositing a complex of bioactive delivery systems onto the hair and scalp.

TABLE 4
Formulation

| Phase | Ingredient (INCI Name) | Function | wt % |
|---|---|---|---|
| A | Deionized Water | Solvent | q.s. to 100 |
| A | Glycerin | Humectant | 4.00 |
| B | Cetearyl Alcohol | Emulsifier/Thickener | 5.00 |
| B | Behentrimonium Methosulfate | Cationic Emulsifier/ | 3.00 |
| | (and) Cetyl Alcohol | Conditioner | |
| B | Simmondsia Chinensis | Emollient | 2.00 |
| | (Jojoba) Seed Oil | | |
| C | Plant-Derived Exosomes | Bioactive Delivery | 2.00 |
| | (from Eclipta prostrata) | Vehicle | |
| C | Nano-Encapsulated Caffeine | Stimulant Active | 3.00 |
| C | Liposomal Curcumin | Antioxidant Active | 0.80 |
| C | Rosmarinus Officinalis | Botanical Active | 1.50 |
| | (Rosemary) Leaf Extract | | |
| C | Phenoxyethanol, Caprylyl | Preservative System | 1.00 |
| | Glycol, Sorbic Acid | | |
| C | Citric Acid | pH Adjuster | As needed |

The plant-derived exosomes in Phase C encapsulate one or more Ayurvedic botanical bioactive materials.

### Manufacturing Procedure:

Phase A Preparation: In a primary vessel, Deionized Water and Glycerin are combined and heated to 80° C.

Phase B Preparation: In a separate vessel, the oil-phase ingredients (Cetearyl Alcohol, Behentrimonium Methosulfate blend, Jojoba Seed Oil) are combined and heated to 80° C. with mixing until all components are melted and uniform.

Emulsification: Phase B is slowly added to Phase A under high-shear homogenization to form a fine, stable oil-in-water emulsion. The emulsion is then switched to propeller mixing and allowed to cool.

Phase C Addition (Cool-Down Phase): When the emulsion has cooled to 40° C. or below, the components of Phase C are added sequentially with gentle mixing. The Plant-Derived Exosomes, Nano-Encapsulated Caffeine, and Liposomal Curcumin are added first, followed by the Rosemary CO2 Extract and the preservative system.

Final Adjustments: The pH is adjusted with Citric Acid to a final target of 4.0 to 5.0.

Final Product Characteristics: The resulting product is a thick, white, opaque cream conditioner. This formulation is designed to deposit a substantive layer of conditioning agents and a trio of bioactive carriers onto the scalp and hair, providing nourishment and stimulation that persists after rinsing.

### Example 19—Conditioner with Botanical Exosomes and Non-Encapsulated Stimulant

A rinse-out hair conditioner is prepared to illustrate a formulation wherein the botanical bioactives are delivered via exosomes, while the stimulant (caffeine) is included in its free, non-encapsulated form directly in the emulsion's water phase.

TABLE 5
Formulation

| Phase | Ingredient (INCI Name) | Function | wt % |
|---|---|---|---|
| A | Deionized Water | Solvent | q.s. to 100 |
| A | Caffeine | Stimulant Active | 0.25 |
| A | Sodium Gluconate | Chelating Agent | 0.20 |
| B | Cetearyl Alcohol | Thickener/Emulsifier | 6.00 |
| B | Brassicamidopropyl | Cationic Conditioner/ | 2.00 |
| | Dimethylamine | Emulsifier | |
| C | Lactic Acid | pH Adjuster/Activator | 0.50 |
| D | Plant-Derived Exosomes | Bioactive Delivery | 2.00 |
| | (Botanical Bioactives | Vehicle | |
| | Payload) | | |
| D | Tocopherol (Vitamin E) | Antioxidant | 0.20 |
| D | Caprylyl Glycol, Phenethyl | Preservative System | 1.00 |
| | Alcohol | | |

The plant-derived exosomes in Phase D encapsulate one or more Ayurvedic botanical bioactive materials.

### Manufacturing Procedure:

Phase A Preparation: In the main vessel, Caffeine and Sodium Gluconate are dissolved in Deionized Water. The mixture is heated to 75° C.

Phase B Preparation: In a separate vessel, the oil-phase ingredients are combined and heated to 75° C. until melted.

Emulsification and Neutralization: Phase B is added to Phase A with homogenization. The batch is cooled to 65° C., and Lactic Acid (Phase C) is added to activate the cationic conditioner.

Phase D Addition (Cool-Down Phase): The emulsion is cooled to 40° C. or below. The suspension of Plant-Derived Exosomes, Tocopherol, and the preservative system are then added with gentle mixing.

Final Product Characteristics: The resulting product is a thick, white cream conditioner. This formulation is designed to provide a dual-action effect: an immediate topical impact from the free caffeine upon application, followed by a more sustained, targeted delivery of botanical bioactives from the exosomes that are deposited on the scalp and hair after rinsing.

### Example 20—Hair Growth Foundation Spray with Encapsulated Botanical Bioactives and Reduced Free Stimulant

This describes the preparation of a hair growth foundation spray. The composition is formulated as a hybrid delivery system, comprising a base material that includes a non-encapsulated stimulant material, and a dispersed phase of plant-derived extracellular vesicles that encapsulate a complex of botanical bioactives.

A hair growth foundation spray is prepared from the ingredients listed in the following table. The formulation is an aqueous solution containing conditioning agents, a non-encapsulated stimulant, and the encapsulated Ayurvedic complex.

TABLE 6
Formulation

| Phase | Ingredient (INCI Name) | Function | wt % |
|---|---|---|---|
| A | Water (Aqua) | Solvent | 68.49 |
| A | Aloe Barbadensis Leaf Juice | Hydrating Agent | 3.99 |
| A | Citric Acid | pH Adjuster | 0.24 |
| B | Lecithin (Sunflower-Derived) | Emulsifying Agent | 0.79 |
| B | Guar Hydroxypropyltrimonium | Conditioning Agent | 0.79 |
| | Chloride | | |
| B | Xanthan Gum | Stabilizer | 0.24 |
| B | Sodium Phytate | Chelating Agent | 0.40 |
| C | Camellia Sinensis Leaf Extract | Stimulant Material | 0.40 |
| | (with Caffeine) | | |
| C | Hydrolyzed Quinoa Protein | Strengthening Agent | 1.20 |
| C | Panthenol (Provitamin B5) | Moisturizing Agent | 0.40 |
| C | Niacinamide (Vitamin B3) | Scalp Barrier Support | 0.40 |
| D | Plant-Derived exosomes | Delivery System | 7.89 |
| D | Leuconostoc/Radish Root | Preservative | 0.64 |
| | Ferment Filtrate | | |
| D | Sodium Benzoate | Preservative | 0.40 |
| D | Fragrance Blend | Fragrance | 0.40 |
| D | Vetiveria Zizanoides Root Oil | Fragrance Fixative | 0.16 |

The plant-derived exosomes in Phase D encapsulate one or more Ayurvedic botanical bioactive materials.

### Manufacturing Procedure:

The composition is prepared using a multi-phase process designed to ensure the stability of the formulation and the integrity of the heat-sensitive components, particularly the extracellular vesicles.

Preparation of the aqueous phase (Phase A): In a primary jacketed mixing vessel equipped with a propeller mixer, the deionized water is added and heated to a controlled temperature of 40° C. Once the temperature is stable, the Aloe Barbadensis Leaf Juice and Citric Acid are added. The mixture is stirred at a moderate speed until all components are fully dissolved and the phase is uniform.

Preparation of the Conditioning Phase (Phase B): In a separate, smaller vessel, the Lecithin, Guar Hydroxypropyltrimonium Chloride, Xanthan Gum, and Sodium Phytate are combined and heated to 50° C. with continuous mixing to form a homogeneous slurry.

Combination of Phases: The heated slurry from Phase B is slowly and steadily added to the primary vessel containing Phase A under continuous, moderate agitation. The mixing continues until the combined batch is homogeneous and slightly thickened. The heat to the vessel jacket is then turned off.

Addition of the Active Phase (Phase C): The batch is allowed to cool under gentle agitation. The temperature is monitored closely until it reaches 35° C. or below. At this temperature, the ingredients of Phase C—Camellia Sinensis Leaf Extract, Hydrolyzed Quinoa Protein, Panthenol, and Niacinamide—are added sequentially, allowing for full incorporation of each ingredient before the next is introduced.

Cool-Down and Vesicle Incorporation (Phase D): The batch continues to cool. Once the temperature is confirmed to be no greater than 40° C., the components of Phase D are added. The Plant-Derived Extracellular Vesicles are added first. This component, at 7.89 wt %, comprises both the lipid vesicle structures and their entire encapsulated payload. The vesicle component is added slowly to the batch under low-shear mixing conditions (e.g., using a paddle mixer at low RPM) to ensure uniform dispersion while preserving the structural integrity of the vesicles.

Finalization: Following the complete incorporation of the vesicles, the remaining preservative and fragrance components of Phase D are added. A sample is taken to measure the final pH, which is adjusted as necessary with additional citric acid or a suitable base to fall within the target range of 4.5 to 5.0. The resulting product is a low-viscosity, non-greasy spray, which is then packaged into sterilized spray bottles.

### Example 21—Hair Growth Foundation Spray with Dual-Carrier Stimulant and Encapsulated Botanical Bioactives

This describes the preparation of a hair growth foundation spray that uses a multi-carrier system for the delivery of its active ingredients. The formulation includes a stimulant material delivered in two forms: a non-encapsulated (“free”) form and a nano-encapsulated form. Concurrently, a complex of Ayurvedic botanical bioactives is delivered exclusively via plant-derived extracellular vesicles. A foundation spray is prepared from the ingredients listed in the following table.

TABLE 7
Formulation

| Phase | Ingredient (INCI Name) | Function | wt % |
|---|---|---|---|
| A | Water (Aqua) | Solvent | 68.24 |
| A | Aloe Barbadensis Leaf Juice | Hydrating Agent | 3.99 |
| A | Citric Acid | pH Adjuster | 0.24 |
| B | Lecithin (Sunflower-Derived) | Emulsifying Agent | 0.79 |
| B | Guar Hydroxypropyltrimonium | Conditioning Agent | 0.79 |
| | Chloride | | |
| B | Xanthan Gum | Stabilizer | 0.24 |
| B | Sodium Phytate | Chelating Agent | 0.40 |
| C | Camellia Sinensis Leaf Extract | Stimulant Material | 0.20 |
| C | Hydrolyzed Quinoa Protein | Strengthening Agent | 1.20 |
| C | Panthenol (Provitamin B5) | Moisturizing Agent | 0.40 |
| C | Niacinamide (Vitamin B3) | Scalp Barrier Support | 0.40 |
| D | Nano-Encapsulated Caffeine | Stimulant Material | 0.50 |
| D | Plant-Derived Exosomes | Delivery System | 7.39 |
| D | Leuconostoc/Radish Root | Preservative | 0.64 |
| | Ferment Filtrate | | |
| D | Sodium Benzoate | Preservative | 0.40 |
| D | Fragrance Blend | Fragrance | 0.40 |
| D | Vetiveria Zizanoides Root Oil | Fragrance Fixative | 0.16 |

The plant-derived exosomes in Phase D encapsulate one or more Ayurvedic botanical bioactive materials.

### Manufacturing Procedure:

The manufacturing process is analogous to that of Example 20, with a modified cool-down phase to accommodate the sequential addition of the multiple carrier systems.

Preparation of the Base Formulation: A base formulation is created by preparing and combining Phases A, B, and a simplified Phase C (containing the non-encapsulated stimulant and other base actives) according to the procedures detailed in Example 20.

Cool-Down and Multi-Carrier Incorporation (Phase D): The batch is cooled to a temperature confirmed to be below 40° C. The delivery components of Phase D are then added sequentially, with gentle mixing after each addition to ensure uniform dispersion.

First, the Nano-Encapsulated Caffeine is added to the batch. This component consists of caffeine encapsulated within a suitable polymeric or lipid-based nanoparticle system.

Second, after the nano-capsules are fully dispersed, the Plant-Derived Extracellular Vesicles are added. This component, at 7.39 wt %, represents the vesicles and their entire encapsulated Ayurvedic payload. This component is added under low-shear conditions.

Finalization: Following the incorporation of both carrier systems, the remaining preservative and fragrance ingredients are added. The pH is checked and adjusted to the 4.5-5.0 range. The final product is a stable, low-viscosity spray containing three distinct forms of active ingredients: free stimulant, nano-encapsulated stimulant, and vesicle-encapsulated botanicals.

### Example 22—Hair Growth Leave-In Treatment with Light Hold and Encapsulated Botanical Bioactives

This describes the preparation of a different product form: a leave-in treatment formulated as a stable oil-in-water emulsion. This composition provides conditioning, light styling hold, and delivers a complex of Ayurvedic botanical bioactives exclusively via plant-derived extracellular vesicles. The leave-in treatment is prepared from the ingredients listed in the following table.

TABLE 8
Formulation

| Phase | Ingredient (INCI Name) | Function | wt % |
|---|---|---|---|
| A | Water (Aqua) | Solvent | 52.13 |
| A | Aloe Barbadensis Leaf Juice | Hydrating Agent | 3.95 |
| A | Glycerin | Humectant | 2.37 |
| A | Camellia Sinensis Leaf Extract | Stimulant/Antioxidant | 2.37 |
| A | Niacinamide | Scalp Barrier Support | 1.58 |
| A | Sodium Hyaluronate | Moisturizing Agent | 0.39 |
| B | VP/VA Copolymer | Film Former/Hold Agent | 3.95 |
| B | Hydrolyzed Quinoa | Strengthening Agent | 1.18 |
| B | Guar Hydroxypropyltrimonium | Conditioning Agent | 0.79 |
| | Chloride | | |
| B | Xanthan Gum | Stabilizer | 0.24 |
| B | Lecithin | Emulsifier | 0.79 |
| C | Caprylic/Capric Triglyceride | Emollient | 7.90 |
| C | Simmondsia Chinensis (Jojoba) | Emollient | 3.95 |
| | Seed Oil | | |
| C | Panthenol | Moisturizing Agent | 1.58 |
| D | Plant-Derived exosomes | Delivery System | 3.86 |
| D | Leuconostoc/Radish Root | Preservative | 0.63 |
| | Ferment Filtrate | | |
| D | Sodium Benzoate | Preservative | 0.39 |
| D | Fragrance Blend | Fragrance | 0.39 |

The plant-derived exosomes in Phase D encapsulate one or more Ayurvedic botanical bioactive materials.

### Manufacturing Procedure:

Preparation of the Aqueous Phase (Phase A): The ingredients of Phase A are combined in a primary vessel and heated to 40° C. with mixing until a clear, uniform solution is formed.

Preparation of the Polymer and Conditioning Phase (Phase B): In a separate vessel, the VP/VA Copolymer, Hydrolyzed Quinoa, Guar Hydroxypropyltrimonium Chloride, Xanthan Gum, and Lecithin are dispersed and hydrated according to supplier specifications. This phase is then added to the primary vessel containing Phase A and mixed thoroughly to form the continuous phase.

Preparation of the Oil Phase (Phase C): In a third vessel, the oil-phase ingredients of Phase C (Caprylic/Capric Triglyceride, Jojoba Seed Oil, and Panthenol) are combined and heated to 35° C. with gentle mixing.

Emulsification: The heated oil phase (C) is slowly added to the combined aqueous phase (A+B) under high-shear homogenization (e.g., using a rotor-stator homogenizer) to form a fine, stable oil-in-water emulsion.

Cool-Down and Vesicle Incorporation (Phase D): The resulting emulsion is switched to low-shear mixing (e.g., a sweep-wall anchor agitator) and is allowed to cool. Once the emulsion temperature is confirmed to be below 40° C., the Plant-Derived Extracellular Vesicles are gently folded into the emulsion. This component, at 3.86 wt %, comprises the vesicles and their entire encapsulated botanical bioactives payload.

Finalization: After the vesicles are uniformly dispersed, the preservatives and fragrance are added. The pH is adjusted to 4.5-5.0. The final product is a creamy lotion that spreads easily, provides conditioning, and leaves a light, flexible hold on the hair after drying.

### Example 23—Hair Growth Leave-In Treatment with a Triple-Carrier System

A leave-in treatment is prepared that exemplifies a triple-carrier system. This composition comprises plant-derived lipid vesicles, liposomes, and ceramide complexes, each encapsulating a different bioactive material. The composition is prepared from the ingredients listed in the following table.

TABLE 9
Formulation

| Phase | Ingredient (INCI Name) | Function | wt % |
|---|---|---|---|
| A | Water (Aqua) | Solvent | 52.44 |
| A | Aloe Barbadensis Leaf Juice | Hydrating Agent | 3.95 |
| A | Glycerin | Humectant | 2.37 |
| A | Niacinamide | Scalp Barrier Support | 1.58 |
| B | VP/VA Copolymer | Film Former/Hold Agent | 3.95 |
| B | Hydrolyzed Quinoa | Strengthening Agent | 1.18 |
| B | Guar Hydroxypropyltrimonium | Conditioning Agent | 0.79 |
| | Chloride | | |
| B | Xanthan Gum | Stabilizer | 0.24 |
| B | Lecithin | Emulsifier | 0.79 |
| C | Caprylic/Capric Triglyceride | Emollient | 7.90 |
| C | Simmondsia Chinensis (Jojoba) | Emollient | 3.95 |
| | Seed Oil | | |
| C | Panthenol | Moisturizing Agent | 1.58 |
| D | Plant-Derived Extracellular | Delivery Vehicle | 1.50 |
| | Vesicles | | |
| D | Liposomes (encapsulating | Delivery Vehicle | 1.00 |
| | Curcumin) | | |
| D | Ceramide Complex (encapsu- | Delivery Vehicle | 1.50 |
| | lating one or more Ayurvedic | | |
| | botanical bioactive | | |
| | materials) | | |
| D | Leuconostoc/Radish Root | Preservative | 0.63 |
| | Ferment Filtrate | | |
| D | Sodium Benzoate | Preservative | 0.39 |
| D | Fragrance Blend | Fragrance | 0.39 |

The plant-derived exosomes in Phase D encapsulate one or more Ayurvedic botanical bioactive materials.

The manufacturing process follows the procedure described in Example 22. During the cool-down phase (Phase D), after the emulsion temperature is below 40° C., the three distinct carrier systems are added. First, the plant-derived extracellular vesicles, encapsulating a payload of Ayurvedic botanical bioactive materials, are added. Second, the liposomes, encapsulating curcumin, are added. Third, the ceramide complex is added. Each carrier system is incorporated under low-shear mixing before the next is added. This formulation strategy allows for the simultaneous delivery of multiple bioactive materials using different encapsulation technologies.

### Example 24—Hair Care System and Method of Use with Dual-Exosome Serum

A multi-product hair care system is provided, designed for synergistic action to promote hair growth and reduce stress-induced hair loss. The system includes a daily serum, a booster mask, a shampoo, and a conditioner. A method of using the system comprises a daily and weekly regimen.

The daily serum is a hair treatment composition comprising a first fraction of lipid vesicles at approximately 3 wt % and a second fraction of lipid vesicles at approximately 4 wt %. The lipid vesicles are plant-derived extracellular vesicles. The first fraction of lipid vesicles encapsulates a stimulant material, such as polyphenols including caffeine and/or epigallocatechin gallate (EGCG). The second fraction of lipid vesicles encapsulates a botanical bioactive material payload comprising a blend of Ayurvedic botanicals selected from a group such as Withania somnifera, Eclipta prostrata, Phyllanthus emblica, Bacopa monnieri, and/or Serenoa serrulata.

The booster mask is a hair treatment composition comprising the second fraction of lipid vesicles (encapsulating the botanical bioactive material) at a concentration of approximately 0.1 wt % to 0.5 wt %. The mask is formulated with film-forming agents to provide prolonged follicular availability of the botanical bioactive material for at least 8 hours and up to 36 hours after application.

The shampoo is a sulfate-free composition comprising rice water and amino acid base with a pH of 4.5 to 5.5, and the conditioner is a composition comprising lightweight, plant-based conditioning agents designed to maintain scalp health and extend the benefits of the serum. The shampoo and/or conditioner can optionally comprise nano-encapsulated caffeine and/or a low concentration of the second fraction of lipid vesicles.

A method of use includes the daily application of the serum to the scalp. One to two times per week, the booster mask is applied to the hair and scalp after shampooing and left on for 5-10 minutes before rinsing. The shampoo and conditioner are used for regular cleansing and conditioning, 3 to 5 times per week. This combined regimen can support a reduction in the appearance of shedding, promote the anagen growth phase, and improve the appearance of hair density and vitality.

### Example 25—Hair Care System and Method of Use with Stimulant-Free Lipid Vesicles

A hair care system is provided wherein the lipid vesicles are reserved exclusively for the delivery of botanical bioactive materials, and the stimulant material is delivered via other means. The system includes a daily serum, a booster mask, a shampoo, a conditioner, and a leave-in composition.

The daily serum is a hair treatment composition comprising a base material and approximately 4 wt % of plant-derived lipid vesicles. The lipid vesicles encapsulate a botanical bioactive material payload. The base material of the serum includes a non-encapsulated stimulant material, such as caffeine, at a concentration of 0.3 wt % to 0.4 wt %.

The booster mask comprises the botanical-encapsulating lipid vesicles at approximately 0.1 wt % and is formulated to extend follicular availability for at least 12 hours.

The shampoo is formulated to be free of lipid vesicles and comprises a rice water and amino acid base. The conditioner is also free of lipid vesicles but contains non-encapsulated bioactives to replenish the scalp.

The optional leave-in composition is a spray or lotion containing nano-encapsulated caffeine to provide an additional source of stimulant material.

The method of use is analogous to that described in Example 24. This system design separates the delivery strategies for different classes of active ingredients.

### Example 26—Hair Care System and Method of Use with Stimulant Delivery in Conditioner

A hair care system is provided that strategically delivers different active-containing lipid vesicles in different products within the regimen. The system includes a daily serum, a booster mask, and a conditioner.

The daily serum is a composition comprising a first fraction of lipid vesicles (Exo-1) encapsulating a stimulant material at 1-3 wt % and a second fraction of lipid vesicles (Exo-2) encapsulating a botanical bioactive material at 2-5 wt %.

The weekly booster mask is a composition that contains only the second fraction of lipid vesicles (Exo-2) at 0.1-1 wt %, focusing the booster treatment on deep nourishment from the botanical payload.

The conditioner is a composition formulated to contain a non-encapsulated stimulant material such as caffeine at 0.1-5 wt %.

A method of use includes daily application of the serum. During washing, after shampooing and rinsing, the conditioner is applied. This step is designed to deliver a supplemental dose of the non-encapsulated stimulant material directly to the scalp and hair after the cleansing step, potentially enhancing its uptake. The booster mask is used weekly in place of the conditioner.

### Example 27—Hair Care System and Method of Use with Non-Encapsulated Stimulant

A hair care system is provided wherein a non-encapsulated stimulant material is used consistently across multiple products, while lipid vesicles are used for targeted botanical delivery. The system includes a serum, a conditioner, and a leave-in composition.

Each of the serum, conditioner, and/or leave-in composition is formulated to include a non-encapsulated stimulant material, such as caffeine, in its base material at a concentration of approximately 0.1 wt % to 0.4 wt %.

The serum, a booster mask, and/or the conditioner are formulated to include plant-derived lipid vesicles. These lipid vesicles, present at concentrations from 0.1 wt % to 7 wt % depending on the product, encapsulate a payload of botanical bioactive materials.

A method of use involves the sequential application of these products. This regimen provides a constant background level of free stimulant material to the scalp, while the lipid vesicles provide a separate, targeted delivery of nourishing botanical compounds.

### ILLUSTRATIVE EMBODIMENTS

The following is a description of various embodiments of the disclosed subject matter. Each embodiment can include one or more of the various features, characteristics, or advantages of the disclosed subject matter. The embodiments are intended to illustrate a few aspects of the disclosed subject matter and should not be considered a comprehensive or exhaustive description of all possible embodiments.
P1. A hair treatment composition comprising: a first fraction of lipid vesicles encapsulating a first material; and a second fraction of lipid vesicles encapsulating a second material; wherein the first material and the second material are different.
P2. The hair treatment composition of P1 wherein the lipid vesicles in the first fraction of lipid vesicles and/or the second fraction of lipid vesicles are extracellular vesicles.
P3. The hair treatment composition of any one of P1 to P2 wherein the lipid vesicles in the first fraction of lipid vesicles and/or the second fraction of lipid vesicles are exosomes.
P4. The hair treatment composition of any one of P1 to P3 wherein the lipid vesicles in the first fraction of lipid vesicles and/or the second fraction of lipid vesicles are liposomes.
P5. The hair treatment composition of any one of P1 to P4 wherein the lipid vesicles in the first fraction of lipid vesicles and/or the second fraction of lipid vesicles are plant-derived lipid vesicles.
P6. The hair treatment composition of any one of P1 to P5 wherein the first material is a stimulant material.
P7. The hair treatment composition of P6 wherein the stimulant material is a polyphenol.
P8. The hair treatment composition of any one of P6 to P7 wherein the stimulant material includes caffeine and/or a green tea catechin.
P9. The hair treatment composition of any one of P1 to P8 wherein the second material is a botanical bioactive material.
P10. The hair treatment composition of P9 wherein the botanical bioactive material is an Ayurvedic botanical bioactive material.
P11. The hair treatment composition of any one of P1 to P10 comprising: no more than approximately 15 wt % of the first fraction of lipid vesicles; and no more than approximately 15 wt % of the second fraction of lipid vesicles.
P12. The hair treatment composition of any one of P1 to P11 comprising: approximately 0.05 to approximately 15 wt % of the first fraction of lipid vesicles; and approximately 0.05 to approximately 15 wt % of the second fraction of lipid vesicles.
P13. The hair treatment composition of any one of P1 to P12 comprising no more than approximately 20 wt % of a total of the first fraction of lipid vesicles and the second fraction of lipid vesicles.
P14. The hair treatment composition of any one of P1 to P13 comprising approximately 0.1 to approximately 20 wt % of a total of the first fraction of lipid vesicles and the second fraction of lipid vesicles.
P15. A hair care system comprising: a daily serum, a booster mask, shampoo, conditioner, and/or a leave-in composition; wherein the daily serum, the booster mask, the shampoo, the conditioner, and/or the leave-in composition comprise the hair treatment composition of any one of P1 to P14.
P16. A hair care system comprising: at least three of a daily serum, a booster mask, shampoo, conditioner, and/or a leave-in composition; wherein the at least three of the daily serum, the booster mask, the shampoo, the conditioner, and/or the leave-in composition each comprise the hair treatment composition of any one of P1 to P14.
P17. A hair treatment composition comprising: a base material; and lipid vesicles encapsulating a botanical bioactive material; wherein the base material includes a non-encapsulated stimulant material.
P18. The hair treatment composition of P17 comprising no more than approximately 10 wt % of the lipid vesicles.
P19. The hair treatment composition of any one of P17 to P18 comprising approximately 0.025 to approximately 10 wt % of the lipid vesicles.
P20. The hair treatment composition of any one of P17 to P19 comprising no more than approximately 2 wt % of the non-encapsulated stimulant material.
P21. The hair treatment composition of any one of P17 to P20 comprising approximately 0.025 to approximately 2 wt % of the non-encapsulated stimulant material.
P22. The hair treatment composition of any one of P17 to P21 wherein the botanical bioactive material is an Ayurvedic botanical bioactive material.
P23. The hair treatment composition of any one of P17 to P22 wherein the non-encapsulated stimulant material includes caffeine and/or a green tea catechin.
P24. A hair care system comprising: a daily serum, a booster mask, shampoo, conditioner, and/or a leave-in composition; wherein the daily serum, the booster mask, the shampoo, the conditioner, and/or the leave-in composition comprise the hair treatment composition of any one of P17 to P23.
P25. A hair care system comprising: at least three of a daily serum, a booster mask, shampoo, conditioner, and/or a leave-in composition; wherein the at least three of the daily serum, the booster mask, the shampoo, the conditioner, and/or the leave-in composition each comprise the hair treatment composition of any one of P17 to P23.
P26. A hair treatment composition comprising: lipid vesicles encapsulating a first bioactive material; liposomes encapsulating a second bioactive material; and ceramide complexes encapsulating a third bioactive material.
P27. The hair treatment composition of P26 wherein the first bioactive material is a stimulant material and/or a botanical bioactive material.
P28. The hair treatment composition of any one of P26 to P27 wherein the first bioactive material is an Ayurvedic botanical bioactive material.
P29. The hair treatment composition of any one of P26 to P28 wherein the second bioactive material is curcumin.
P30. The hair treatment composition of any one of P26 to P29 wherein the third bioactive material is an Ayurvedic botanical bioactive material.
P31. A method of manufacturing a hair treatment composition comprising: preparing a base material; cooling the base material to a temperature of no more than approximately 45° C. to form a cooled base material; and adding lipid vesicles to the cooled base material, the lipid vesicles encapsulating a bioactive material.
P32. The method of P31 wherein the lipid vesicles are plant-derived extracellular vesicles.
P33. The method of any one of P31 to P32 comprising adjusting the pH of the base material to approximately 4.5 to approximately 5.5.
P34. The method of any one of P31 to P33 comprising packaging the hair treatment composition in an airless container.
P35. A hair shampoo composition comprising: a sulfate-free surfactant system; and plant-derived lipid vesicles encapsulating a bioactive material; wherein the hair shampoo composition has a pH of approximately 4.0 to approximately 6.0.
P36. The hair shampoo composition of P35 wherein the plant-derived lipid vesicles are nanovesicles.
P37. The hair shampoo composition of any one of P35 to P36 wherein the bioactive material is a botanical bioactive material.
P38. A hair treatment composition comprising: approximately 0.025 to approximately 5 wt % of plant-derived extracellular vesicles encapsulating a botanical bioactive material; wherein the hair treatment composition provides at least 8 hours of follicular availability of the botanical bioactive material following topical application.
P39. A hair shampoo composition comprising: a rice water extract; and amino acids including cysteine, methionine, arginine, lysine, serine, glycine, proline, alanine, and/or glutamic acid.
P40. The hair shampoo composition of P39 wherein the amino acids mimic a natural keratin amino acid profile of human hair.
P41. The hair shampoo composition of P39 comprising 0.01 wt % to 5 wt % of the amino acids.
P42. The hair shampoo composition of P39 comprising a botanical bioactive material.
P43. A hair care system comprising: a daily serum comprising plant-derived lipid vesicles encapsulating botanical bioactive materials; a booster mask comprising plant-derived lipid vesicles encapsulating the botanical bioactive materials; and a shampoo and a conditioner both of which are free of lipid vesicles and comprise non-encapsulated forms of the botanical bioactive materials; wherein the shampoo comprises a rice water extract and amino acids.

### General Terminology and Interpretative Conventions

The articles “the,” “a,” and “an” shall be interpreted as referring to both singular and plural forms. Additionally, unless preceded by the word “either” or similar language indicating exclusivity, the term “or” should be interpreted inclusively (for example, “x or y” refers to one or both x and y).

The term “and/or” shall be interpreted inclusively; for example, “x and/or y” refers to either x, y, or both. When “and/or” or “or” is used to connect three or more items, the phrase shall be interpreted to encompass any individual item, all items collectively, or any combination of the items.

The phrase “based on” shall be interpreted to mean an open set of conditions unless it is explicitly limited (for example, based on only a given condition). For instance, if a step is described as being based on a particular condition, it may depend on both the stated condition and additional unstated conditions.

The term “can,” when used as an auxiliary verb, indicates that the described subject matter optionally has the feature, aspect, ability, capacity, or the like, but it is not required in any given instance.

The terms “have,” “having,” “contain,” “containing,” “include,” “including,” and “characterized by” shall be construed as synonymous with “comprise” and “comprising”—that is, these terms are inclusive or open-ended and do not preclude additional, unrecited subject matter. The use of these terms shall also be interpreted as providing disclosure and support for narrower alternative embodiments in which these terms are substituted with “consisting of,” “consisting of the recited subject matter plus impurities and/or trace amounts of other materials,” or “consisting essentially of.”

It shall be understood that features described in separate embodiments can be combined as a single embodiment. Similarly, features described together in a single embodiment can be implemented separately or in various subcombinations across multiple embodiments. Additionally, although certain features may be initially presented or claimed as part of specific combinations, it is possible to remove one or more features from a claimed combination so that the claim pertains to a subcombination or a variation thereof.

Numerous aspects or features are described as optional, often indicated by terms such as “can” or similar expressions. This document does not detail every possible combination or permutation arising from selecting among these optional elements. Nevertheless, all such combinations and permutations are considered expressly disclosed herein. For instance, an item described with three optional aspects may be embodied in seven distinct configurations: any single aspect, any pair of aspects, or all three aspects incorporated together.

The methods described in this document shall not be interpreted to require steps to be performed in a particular order unless explicitly indicated or if it is impossible to do otherwise. The methods should be understood to provide support or basis for the steps to be carried out in any sequence.

The configurations presented in this document are provided as examples and do not encompass all possible implementations within the scope of the claims. The term “example” is used to indicate an instance or illustration, without implying that it is preferred or superior to other possibilities.

Unless otherwise indicated, all numerical values or expressions regarding dimensions, physical characteristics, or similar parameters in the specification (excluding the claims) shall be interpreted as qualified by the term “approximately.” Additionally, if there is an absence of functional, qualitative, or other interpretative guidelines, each numerical value identified as “approximately” within the specification and claims shall be interpreted based on the stated number of significant digits and/or standard rounding conventions, without limiting the application of the doctrine of equivalents to the claims.

All disclosed ranges shall be interpreted as encompassing, and providing support for, claims that specify any subranges or individual values contained within each stated range. For instance, if a range is described as 1 to 10, it is intended to include and support claims directed to all subranges and individual values falling between, and including, the minimum value of 1 and the maximum value of 10. This includes all subranges commencing at a value of 1 or higher and concluding at a value of 10 or lower (e.g., 5.5 to 10, 2.34 to 3.56), as well as any individual values from 1 to 10 (e.g., 3, 5.8, 9.9994). The values in the range may be recited independently, as a minimum value (e.g., at least 5.8), or as a maximum value (e.g., no more than 9.9994).

All disclosed numerical values shall be interpreted as variable within a range of 0-100% in either direction. This interpretation provides support for claims referencing these values, whether stated individually or as a minimum or maximum (e.g., at least <value> or no more than <value>), as well as any ranges or subranges that can be derived from such values. For instance, a specified value of 8 should be considered as extending from 0-16 (reflecting 100% variability in both directions) and supports claims referring to the entire range (e.g., 0-16), any subrange within this interval (e.g., 2-12.5), or any individual value within the range (e.g., 15.2), including its use as a minimum value (e.g., at least 4.3) or a maximum value (e.g., no more than 12.4).

The terms in the claims shall be interpreted according to their ordinary and customary meanings, as established by relevant entries in widely recognized general or technical dictionaries and by commonly accepted meanings within the pertinent field. The interpretation should reflect the broadest meaning derived from any single source or from a combination of these sources (e.g., by synthesizing multiple dictionary definitions to arrive at the most comprehensive understanding), except under the following circumstances: (a) if a term is employed in a manner that extends beyond its ordinary usage, it should be accorded its customary meaning along with the broader meaning or (b) if a term is expressly defined within the document by language such as “as used in this document <term> shall mean,” “this term means,” “this term is defined as,” or “for the purposes of this disclosure this term shall mean.” References to specific examples, use of “i.e.,” or use of the word “invention” are not intended to invoke exception (b) or to otherwise narrow the scope of the claim terms. Except where exception (b) applies, nothing in this document should be construed as a disclaimer or limitation of claim scope.

The limitations in the claims shall not be interpreted as invoking 35 U.S.C. 112 (f) unless the claim specifically includes the terms “means for” or “step for.”

The subject matter recited in the claims is not coextensive with and should not be interpreted as coextensive with any embodiment, feature, or combination of features described or illustrated in this document. This is the case even if only one version of a feature or combination is depicted and discussed.

### Composition Related Terminology and Interpretative Conventions

Values expressed as a percentage, parts of, or a ratio are by weight unless expressly stated otherwise.

The description of a group or class of materials as suitable or preferred for a given purpose shall be understood as disclosing that a single member of the group or class or a mixture of any two or more members of the group or class are equally suitable or preferred.

The description of constituents in chemical terms refers to the constituents: (a) at the time of addition to any combination specified in the description (e.g., the formal or analytical concentration of a salt that dissociates in solution) and/or (b) generated in situ by chemical reactions with other constituents. The description of the constituents does not preclude other chemical interactions among the constituents of a mixture once mixed unless expressly stated otherwise.

The description of materials in ionic form additionally implies the presence of sufficient counter ions to produce electrical neutrality for the composition.

### INCORPORATION BY REFERENCE

The entire content of each document listed below is incorporated by reference into this document (the documents below are collectively referred to as the “incorporated documents”). If the same term is used in both this document and one or more of the incorporated documents, then it should be interpreted to have the broadest meaning imparted by any one or combination of these sources unless the term has been explicitly defined to have a different meaning in this document. If there is an inconsistency between any incorporated document and this document, then this document shall govern. The incorporated subject matter should not be used to limit or narrow the scope of the explicitly recited or depicted subject matter.

Benefit/priority patent documents incorporated by reference:
U.S. Prov. App. No. 63/699,034, titled “Hair Care Products,” filed on 25 Sep. 2025.

1. A hair treatment composition comprising:
a first fraction of lipid vesicles encapsulating a first material; and
a second fraction of lipid vesicles encapsulating a second material;
wherein the first material and the second material are different.

2. The hair treatment composition of claim 1 wherein the lipid vesicles in the first fraction of lipid vesicles and/or the second fraction of lipid vesicles are extracellular vesicles.

3. The hair treatment composition of claim 1 wherein the lipid vesicles in the first fraction of lipid vesicles and/or the second fraction of lipid vesicles are plant-derived lipid vesicles.

4. The hair treatment composition of claim 1 wherein the first material is a stimulant material.

5. The hair treatment composition of claim 4 wherein the stimulant material includes caffeine and/or a green tea catechin.

6. The hair treatment composition of claim 1 wherein the second material is a botanical bioactive material.

7. The hair treatment composition of claim 6 wherein the botanical bioactive material is an Ayurvedic botanical bioactive material.

8. The hair treatment composition of claim 1 comprising:
no more than approximately 15 wt % of the first fraction of lipid vesicles; and
no more than approximately 15 wt % of the second fraction of lipid vesicles.

9. The hair treatment composition of claim 1 comprising no more than approximately 20 wt % of a total of the first fraction of lipid vesicles and the second fraction of lipid vesicles.

10. A hair treatment composition comprising:
a base material; and
lipid vesicles encapsulating a botanical bioactive material;
wherein the base material includes a non-encapsulated stimulant material.

11. The hair treatment composition of claim 10 comprising no more than approximately 10 wt % of the lipid vesicles.

12. The hair treatment composition of claim 10 comprising no more than approximately 2 wt % of the non-encapsulated stimulant material.

13. The hair treatment composition of claim 10 wherein the botanical bioactive material is an Ayurvedic botanical bioactive material.

14. The hair treatment composition of claim 10 wherein the non-encapsulated stimulant material includes caffeine and/or a green tea catechin.

15. A hair care system comprising:
a daily serum, a booster mask, shampoo, conditioner, and/or a leave-in composition;
wherein the daily serum, the booster mask, the shampoo, the conditioner, and/or the leave-in composition comprise the hair treatment composition of claim 10.

16. The hair care system of claim 15 wherein the daily serum and the booster mask include the hair treatment composition.

17. A hair shampoo composition comprising:
a rice water extract; and
amino acids including cysteine, methionine, arginine, lysine, serine, glycine, proline, alanine, and/or glutamic acid.

18. The hair shampoo composition of claim 17 wherein the amino acids mimic a natural keratin amino acid profile of human hair.

19. The hair shampoo composition of claim 17 comprising 0.01 wt % to 5 wt % of the amino acids.

20. The hair shampoo composition of claim 17 comprising a botanical bioactive material.
